(19) United States
(12) Patent Application Publication
(10) Pub. No.: US 2020/0347407 A1
(43) Pub. Date: Nov. 5, 2020

(54) SPLIT SINGLE-BASE GENE EDITING SYSTEMS AND APPLICATION THEREOF

(71) Applicants: EAST CHINA NORMAL UNIVERSITY, Shanghai (CN); BIORAY LABORATORIES INC., Shanghai (CN)

(72) Inventors: Dali LI, Shanghai (CN); Xiaohui ZHANG, Shanghai (CN); Liren WANG, Shanghai (CN); Biyun ZHU, Shanghai (CN); Liang CHEN, Shanghai (CN); Mingyao LIU, Shanghai (CN)

(21) Appl. No.: 16/955,453

(22) PCT Filed: Dec. 18, 2018
(86) PCT No.: PCT/CN2018/121779

§ 371 Date: Jun. 18, 2020

(30) Foreign Application Priority Data
Dec. 18, 2017 (CN) 201711368309.6

(51) Int. Cl. C12N 15/86; C12N 15/113; C12N 9/22; C12N 9/78

(52) U.S. Cl. CPC C12N 15/86; C12N 15/113; C12N 9/22; C12N 2510/00; C12Y 305/04001; C12Y 305/04004; C12N 9/78

(57) ABSTRACT

Provided are two split single-base gene editing systems. Intein-mediated split BE3, saKKH-BE3 and ABE7.10 are separately developed by using the existing protein structure information of spCas9 and saCas9 and splitting methods thereof, and have targeted gene mutation efficiency equivalent to that of unsplit BE3, saKKH-BE3 and ABE7.10 working system, thereby making possible package into an AAV for delivery.

### TECHNICAL FIELD

The present invention relates to the field of biotechnology, in particular, to a split-single base gene editing system and the application thereof.

### BACKGROUND

Since 2013, a new generation of gene editing technology represented by CRISPR/Cas9 has entered various labs in the field of biology and is changing the traditional methods of gene manipulation.

At present, the single-base gene editing technology has been reported to be used for efficient gene mutation or repair in genome, generation of disease animal model and gene therapy. Among the found single-base gene editing tools, BE3, SaKKH-BE3, and ABE7.10, are the most widely used. However, BE3, SaKKH-BE3, and ABE7.10 has a length of 5.1 kb, 4.3 kb, and 5.3 kb, respectively. With the addition of the promoter and polyA elements, it has exceeded the 4.7 kb packaging limit of the adeno-associated virus (AAV), which prevents them from being packaged into AAV for delivery and hinders the widespread use in gene editing, gene therapy and clinical practice.

Therefore, there is an urgent need in the art to develop a split single-base gene editing system that can maintain a considerable targeted gene mutation efficiency.

### SUMMARY OF THE INVENTION

The object of the present invention is to provide a split single-base gene editing system that can maintain a considerable targeted gene mutation efficiency.

The object of the present invention is also to provide a split single-base gene editing system (BE3, SaKKH-BE3, ABE7.10), which is split into N-terminal and C-terminal, respectively, the length of which is less than the adeno-associated virus (AAV) packaging limit of 4.7 Kb and can be packaged and delivered with AAV, further expanding the single-base gene editing technology, especially in the field of gene therapy.

In a first aspect of the present invention, it provides a nucleic acid construct combination comprising a first nucleic acid construct and a second nucleic acid construct, wherein the first nucleic acid construct has a structure of Formula I from 5′-3′:

P1-X1-X2-X3-Z-X4 (I);

wherein P1 is a first promoter sequence;

X1 is a coding sequence of cytosine deaminase or a coding sequence of adenosine deaminase;

X2 is an optional linker sequence;

X3 is a coding sequence of the N-terminal fragment of Cas9 nuclease or a coding sequence of the N-terminal fragment of SaKKH nuclease (Cas9n-N or SaKKH-N);

Z is a coding sequence of the N-terminal fragment of a first fusion peptide;

X4 is a polyA sequence;

the second nucleic acid construct has a structure of Formula II from 5′-3′:

P2-Z-Y1-Y2-Y3 (II);

wherein P2 is a second promoter sequence;
Z is a coding sequence of the C-terminal fragment of a first fusion peptide;
Y1 is a coding sequence of the C-terminal fragment of Cas9 nuclease or a coding sequence of the C-terminal fragment of SaKKH nuclease (Cas9n-C or SaKKH-C);
Y2 is a coding sequence of UGI or none;
Y3 is a polyA sequence;

and each “-” is independently a bond or a nucleotide linker sequence.

In another preferred embodiment, if X1 is a coding sequence of adenosine deaminase, Y2 is none.

In another preferred embodiment, the first promoter and the second promoter are each independently selected from the group consisting of: a CAG promoter, CMV promoter, and a combination thereof.

In another preferred embodiment, the first promoter sequence comprises a CMV promoter.

In another preferred embodiment, the second promoter sequence comprises a CMV promoter.

In another preferred embodiment, the linker sequence includes XTEN, GGS, (GGS) 3, (GGS) 7.

In another preferred embodiment, the cytosine deaminase includes Apobec1.

In another preferred embodiment, the adenosine deaminase is derived from a bacteria, human, rat, and/or mouse.

In another preferred embodiment, the Cas9 nuclease is selected from the group consisting of Cas9, Cas9n, and a combination thereof.

In another preferred embodiment, the N-terminal fragment of Cas9n is amino acids 2-573 of Cas9n nuclease (Accession Number (Gene ID): 2828055).

In another preferred embodiment, the C-terminal fragment of Cas9n is amino acids 574-1368 of Cas9n nuclease (Accession Number (Gene ID): 2828055).

In another preferred embodiment, the SaKKH nuclease is from Staphylococcus.

In another preferred embodiment, the N-terminal fragment of SaKKH is amino acids 2-739 of SaKKH nuclease (Accession Number (Gene ID): 2828033).

In another preferred embodiment, the C-terminal fragment of SaKKH is amino acids 740-1053 of SaKKH nuclease.

In another preferred embodiment, the origin of the X3 element is selected from the group consisting of Streptococcus pyogenes, Staphylococcus aureus, and a combination thereof.

In another preferred embodiment, in the X3 element, the mutation site is at position D10A of Cas9n-N(SEQ ID NO.: 1).

In another preferred embodiment, in the X3 element, the mutation site is at position D10A of SaKKH-N(SEQ ID NO.: 2).

In another preferred embodiment, in the Y1 element, there is no mutation site in Cas9n-C (SEQ ID NO.: 4).

In another preferred embodiment, in the Y1 element, the mutation site is at position E782K/N968K/R1015H of SaKKH-C(SEQ ID NO.: 3).

In another preferred embodiment, the polyA sequence is each independently selected from the group consisting of BGH polyA, SV40 polyA, and a combination thereof.

In another preferred embodiment, the length of the first nucleic acid construct is ≤4.7 kb, preferably, ≤4.5 kb, and more preferably, 3.0-4.5 kb.

In another preferred embodiment, the length of the second nucleic acid construct is ≤4.7 kb, preferably, ≤4.5 kb, and more preferably, 3.0-4.5 kb.

In another preferred embodiment, the N-terminal fragment of the first fusion peptide and the C-terminal fragment of the first fusion peptide together constitute an active first fusion peptide.

In another preferred embodiment, the first fusion peptide is selected from the group consisting of intein, FRB/FKBP, DmC/FKBP, ABI/PYL, and a combination thereof.

In another preferred embodiment, the N-terminal fragment of the first fusion peptide is amino acids 2-103 of the fusion peptide (such as an intein).

In another preferred embodiment, the C-terminal fragment of the first fusion peptide is amino acids 104-137 of the fusion peptide (e.g., intein).

In a second aspect of the present invention, it provides a vector combination, comprising a first vector and a second vector, the first vector contains a first nucleic acid construct, the second vector contains the second nucleic acid construct, the first nucleic acid construct and the second nucleic acid construct are as defined in the first aspect of the present invention.

In another preferred embodiment, the first vector and the second vector are viral vectors.

In another preferred embodiment, the first vector and the second vector are AAV viral vectors.

In another preferred embodiment, in the first vector, the first construct is located in an expression cassette with inverted terminal repeats at both ends of the first vector.

In another preferred embodiment, in the second vector, the second construct is located in an expression cassette with inverted terminal repeats at both ends of the second vector.

In a third aspect of the present invention, it provides a genetically engineered cell wherein the cell is transformed by the construct according to the first aspect of the present invention; or transformed or transfected by the vector combination according to the second aspect of the present invention.

In another preferred embodiment, the vector combination includes a first viral vector and a second viral vector, preferably, the first viral vector and the second viral vector are both AAV viral vectors.

In another preferred embodiment, the genetically engineered cell is a prokaryotic cell or a eukaryotic cell.

In another preferred embodiment, the prokaryotic cell includes: E. coli.

In another preferred embodiment, the eukaryotic cell is selected from the group consisting of a yeast cell, plant cell, mammalian cell (such as a HEK293T cell), human cell, and a combination thereof.

In another preferred embodiment, the genetically engineered cell is prepared by the first viral vector and the second viral vector through viruses. In a fourth aspect of the present invention, it provides a method for gene editing in a cell, comprising the steps of:

(i) providing a cell and a first vector and a second vector, wherein the first vector contains a first nucleic acid construct, the second vector contains a second nucleic acid construct, the first nucleic acid construct and the second nucleic acid construct are as defined in the first aspect of the present invention, and both the first vector and the second vector are viral vectors;

(ii) infecting the cell with the viral vector, thereby performing gene editing in the cell.

In another preferred embodiment, in step (ii), the method further comprises simultaneously infecting the cell with a third vector encoding gRNA.

In another preferred embodiment, the first vector and/or the second vector.

In another preferred embodiment, the method is non-diagnostic and non-therapeutic.

In another preferred embodiment, the gene editing comprises: site-specific cleavage, site-specific insertion, and site-specific recombination.

In another preferred embodiment, the cell is from the following species: a human, non-human mammal, poultry, plant.

In another preferred embodiment, the non-human mammal includes a rodent (such as a mouse, rat, rabbit), cow, pig, sheep, horse, dog, cat, and non-human primate (such as a monkey).

In another preferred embodiment, the cell includes: a somatic cell, stem cell, germ cell, non-dividing cell, and a combination thereof.

In another preferred embodiment, the cell includes: a kidney cell, epithelial cell, endothelial cell, and a combination thereof.

In a fifth aspect of the present invention, it provides a kit, comprising:

(a1) a first container, and a first vector located in the first container; and

(b1) a second container, and a second vector located in the second container.

In another preferred embodiment, the first container and the second container are different containers.

In another preferred embodiment, the kit further includes instructions that describe a method of infecting a cell with the first vector and the second vector to perform gene editing in the cell.

In another preferred embodiment, the kit further contains (cl) a third container, and a third container containing a third vector encoding gRNA.

In another preferred embodiment, the first vector and/or the second vector.

It should be understood that, within the scope of the present invention, the technical features specifically described above and below (such as the Examples) can be combined with each other, thereby constituting a new or preferred technical solution which needs not be described one by one.

### DESCRIPTION OF DRAWINGS

FIG. 1 shows a schematic diagram of BE3 and split-BE3(BE3-N, BE3-C). Wherein CMV: promoter; U6: human derived U6 promoter; Apobec1: cytosine deaminase; XTEN: linker; SpCas9n: D10A mutant nicked SpCas9; SpCas9(D10A)(2-573) represents amino acids 2-573 of SpCas9 with a nick of D10A mutation; SpCas9(D10A)(574-1368) represents amino acids 574-1368 of SpCas9 with a nick of D10A mutation; UGI: uracil glycosidase inhibitor; BGH:PolyA sequence.

FIG. 2 shows a schematic diagram of SaKKH-BE3 and the split SaKKH-BE3. Wherein CMV: promoter; U6: human derived U6 promoter; Apobec1: cytosine deaminase; XTEN: linker; SaCas9n(KKH): in addition to the D10A mutation, it also contains the E782K/N968K/R1015H amino acid mutation;

SaCas9(D10A)(2-739) represents amino acids 2-739 of SpCas9 with a nick of D10A mutation; SaCas9(D10A)(740-1053 represents amino acids 740-1053 of SpCas9 with a nick of D10A mutation; UGI: uracil glycosidase inhibitor; BGH: PolyA sequence.

FIG. 3 shows a schematic diagram of ABE7.10 and split-ABE7.10 (ABE7.10-N, ABE7.10-C). Wherein CMV: promoter; U6: human derived U6 promoter; Apobec1: adenosine deaminase; XTEN: linker; SpCas9n: D10A mutated nicked SpCas9; SpCas9 (D10A) (2-573) represents amino acids 2-573 of SpCas9 with a nick of D10A mutation; SpCas9 (D10A) (574-1368) represents amino acids 574-1368 of SpCas9 with a nick of D10A mutation; BGH: PolyA sequence.

FIG. 4 a schematic diagram of U6-spsgRNA-EF1α-GFP. Wherein U6: human derived U6 promoter; sg: represents the insertable SpCas9 target; EF1α: promoter: EGFP: Enhanced Green Fluorecence Protein; PA: PolyA sequence.

FIG. 5 shows a schematic diagram of U6-sasgRNA-EF1α-GFP. Wherein U6: human derived U6 promoter; sg: represents the insertable SaCas9 target; EF1α: promoter: EGFP: Enhanced Green Fluorecence Protein; PA: PolyA sequence.

FIG. 6 shows a list of tested targets.

FIG. 7 shows the comparison of the mutation efficiency of BE3 and split BE3 for endogenous gene targets EMX1 site1, EMX1 site2, RNF2 site1. Among them, the ordinate: representing the percentage of single base C to T mutation; the abscissa: representing C at different positions of 3 endogenous targets of EMX1 site1, EMX1 site2, RNF2 site1.

FIG. 8 shows the comparison of the mutation efficiency of SaKKH and the split SaKKH for the endogenous genes EMX1 site3 and FANCF site1. Among them, the ordinate: represents the percentage of single base C to T mutation; the abscissa: represents C at different positions of 2 endogenous targets of EMX1 site3, FANCF site1.

FIG. 9 shows the comparison of the mutation efficiency of ABE7.10 and split ABE7.10 for the endogenous gene target EMX1 site1. Among them, the ordinate: represents the percentage of single base A to G mutation; the abscissa: represents C at different positions of an endogenous target of EMX1 site1.

### DETAILED DESCRIPTION OF INVENTION

After extensive and intensive research, the inventors has first time found that intein-mediated resolution of BE3, SaKKH-BE3 and ABE7.10 were developed respectively using the existing protein structure information of spCas9 and saCas9 and their splits methods, and the split BE3, SaKKH, ABE7.10 has a higher target gene mutation efficiency, which is comparable to the target gene mutation efficiency of the unsplit BE3, SaKKH, ABE7.10 working system, providing the possibility of packaging into AAV for delivery, and promoting its wide application in gene editing, gene therapy and clinical. On this basis, the inventor has completed the present invention.

BE3 Working System

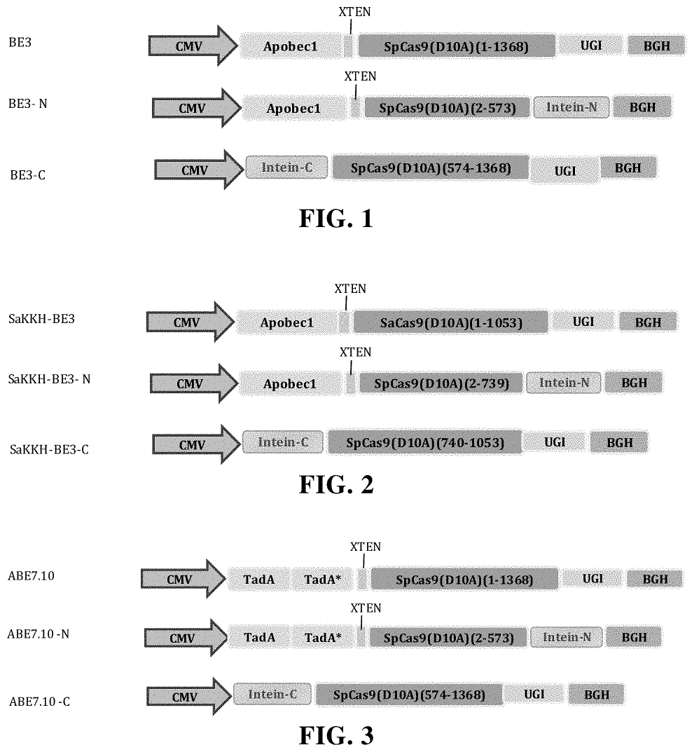

BE3, that is Base editor 3, which is formed by fusion of cytosine deaminase and spCas9 (spCas9n) with a D10A mutation derived from Streptococcus pyogenes. It uses NGG as PAM and recognizes and specifically binds DNA and a single base mutation from C to T is achieved at positions 16-19 upstream of NGG (FIG. 1).

SaKKH-BE3 Working System

SaKKH is formed by fusion of cytosine deaminase and SaCas9 with a D10A mutation and saCas9 with E782K/N968K/R1015H mutation from Staphylococcus aureus with NNNRRT (R=A or G) as PAM and recognize and specifically bind DNA and realize C to T single base mutation at positions 11-16 upstream of PAM (FIG. 2).

ABE7.10 Working System

ABE7.10, which is formed by fusion of adenosine deaminase with spCas9 (spCas9n) with a D10A mutation derived from Streptococcus pyogenes. It uses NGG as PAM and recognizes and specifically binds DNA and realizes a single-base mutation from A to G at positions 16-19 upstream of NGG (FIG. 3).

Self-Cleaving Protein

Self-cleaving protein 2A is a class of 18-22 amino acid peptides. When it connects two or more proteins, its translated protein product can be cleaved between glycine and proline(Asp-Val/Ile-Glu-X-Asn-Pro-Gly-Pro) at highly conserved C-terminal of 2A. Therefore, the protein products at both ends of 2A can be functioned independently. The self-cleaving protein used in this invention is T2A derived from tetrahymenops β-larvae virus (Thosea asignavirus). There are also F2A from foot-and-mouth disease virus, E2A from horse rhinitis virus, and P2A from swine Jieshen 1 virus.

In a preferred embodiment, the self-cleaving protein is a 2A sequence. The 2A sequence is from the virus and is a short peptide of 18-22 amino acids. It expresses multiple proteins in an open reading frame through self-splicing, and the self-splicing efficiency is almost 100%. Commonly used are T2A, P2A, F2A, E2A.

Fusion Peptide

In the present invention, the fusion peptide is not particularly limited, and a preferred fusion peptide is selected from the group consisting of intein, FRB/FKBP, DmC/FKBP, ABI/PYL, and a combination thereof.

In a preferred embodiment, the fusion peptide is an intein. Intein is a naturally occurring intermediate sequence, which can catalyze protein splicing reaction to make inteins and flanking peptides into tandems with natural peptide bonds. Inteins control enzyme activity at specific times based on splicing reactions and coupling of peptides from different sources.

Construct Combination of the Present Invention

The invention provides a nucleic acid construct combination, including a first construct and a second construct. It uses the existing protein structure information of spCas9 or saCas9 and their split methods, intein-mediated split BE3, SaKKH-BE3 and the split BE3, SaKKH-BE3 and ABE7.10 were developed respectively. Among them, the split BE3 and SaKKH-BE3 nucleic acid construct can achieve the C mutation to T in the endogenous gene target, which is equivalent to the targeted gene mutation efficiency of the unsplit BE3 and SaKKH-BE3 working systems. Splitting the ABE7.10 nucleic acid construct can achieve the A mutation to G in the endogenous gene target, compared with the unsplit ABE7.10, the efficiency is slightly lower. The split-type BE3, SaKKH-BE3, and ABE7.10 provide the possibility of being packaged into AAV for delivery, and promote its wide application in base editing, gene therapy and clinical. The construct combination of the invention is as described in the first aspect of the invention.

The various elements used in the construct combination of the invention are known in the field, so those technician in the field can use conventional methods, such as PCR method, fully artificial chemical synthesis method, and enzyme digestion method to obtain the corresponding elements. These elements were ligased together by well-known DNA ligation techniques and then the construct combination of the invention is formed.

Specifically, the construction, verification process and application of the construct combination in the invention are as follows:

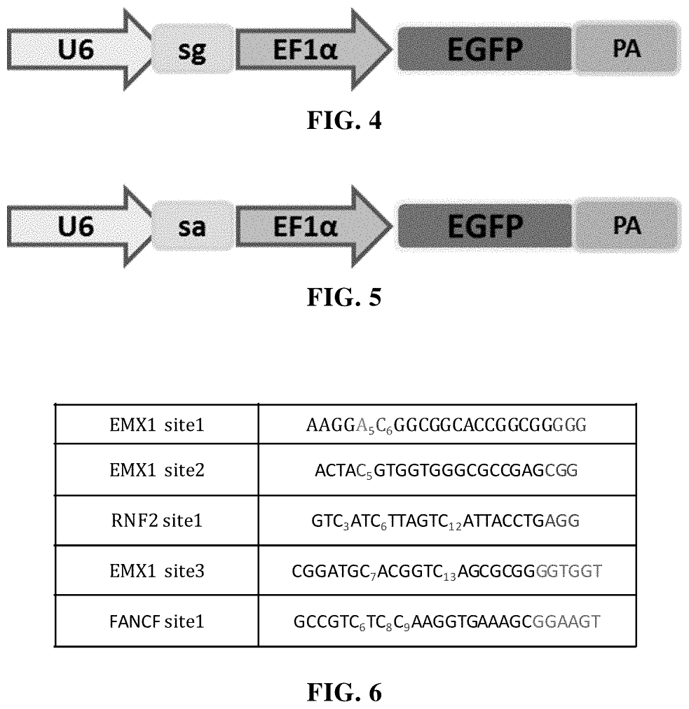

1. According to the human EMX1, RNF2, FANCF gene, BE3, SaKKH-BE3 and ABE7.10 target sequence were designed respectively (FIG. 6), and its sgRNA oligo was designed according to its target (Table 1). According to structure information of Spcas9 and saCas9, using intein as fusion linker peptide, the split BE3 was designed, Splitting occurs between amino acids 573 and 574 of Spcas9, that is BE3-N, BE3-C (FIG. 1), at the same time, the split SaKKH-BE3 was designed and splitting occurs between amino acids 739 and 740 of Sacas9 (FIG. 2), that is SaKKH-BE3-N and SaKKH-BE3-C (FIG. 2). At the same time, the split ABE7.10 was designed, splitting occurs between amino acids 573 and 574 of Spcas9, namely ABE7.10-N, ABE7.10-C (FIG. 3). Simultaneously the sgRNA vector U6-spsgRNA-EF1α-GFP with spCas9 sacffold (FIG. 4) was designed and expressed and the sgRNA vector U6-sasgRNA-EF1α-GFP with saCas9 sacffold (FIG. 5) was expressed. At the same time, according to the working principle of CRISPR/Cas9, the plasmids in FIG. 4 and FIG. 5 were digested with BbsI, and then ligased to the corresponding annealed sgRNA oligo (Table-1).

2. The above plasmids in sequence was constructed.

3. Comparing the fixed-point editing efficiency of C to T for BE3 and BE3-N/BE3-C, SaKKH-BE3 and SaKKH-BE3-N/SaKKH-BE3-C on human endogenous gene targets (FIG. 6); comparing the fixed-point editing efficiency of A to G for ABE7.10 and ABE7.10-N/ABE7.10-C on human endogenous gene targets (FIG. 6).

That is, the following combination was co-transfected with 293T,

BE3: U6-sgRNA-EF1α-GFP=750 ng:250 ng,

BE3-N: BE3-C: U6-sgRNA-EF1α-GFP=375 ng:375 ng:250 ng

SaKKH-BE3: U6-sgRNA-EF1α-GFP=750 ng:250 ng,

SaKKH-BE3-N: SaKKH-BE3-C: U6-sgRNA-EF1α-GFP=375 ng:375 ng:250 ng

ABE7.10: U6-sgRNA-EF1α-GFP=750 ng:250 ng,

ABE7.10-N: ABE7.10-C: U6-sgRNA-EF1α-GFP=375 ng:375 ng:250 ng

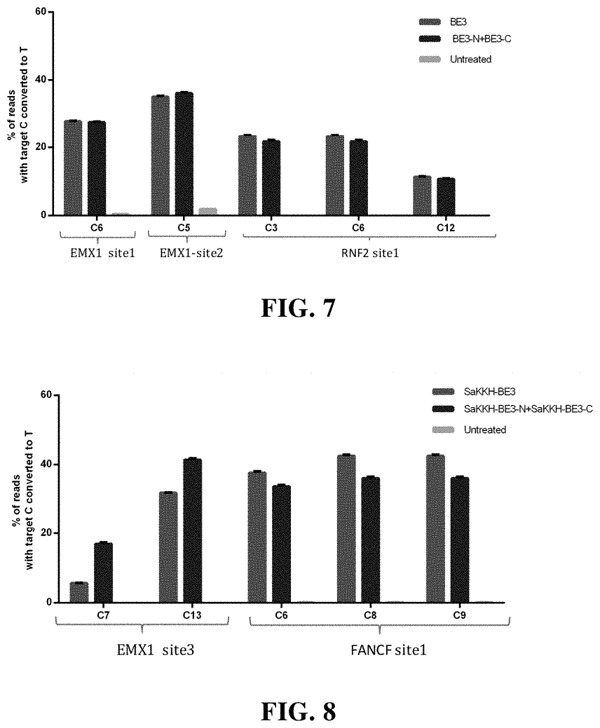

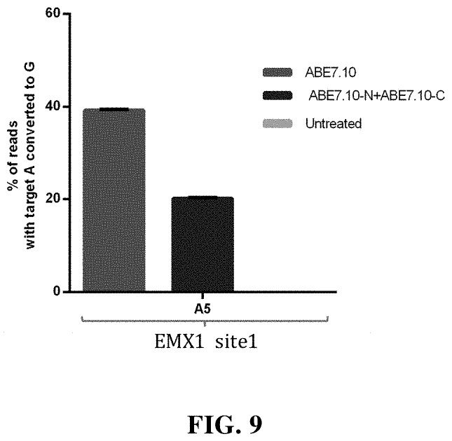

72 h after transfection, GFP positive cells were sorted by flow cytometry. According to the instructions of the HITOM kit (Nuohe Zhiyuan item number: PT026), after performing PCR using the first round of PCR identification primers (Table-2), and then the second round of PCR library construction is completed, high-throughput sequencing was performed, comparing the editing efficiency of endogenous targets for BE3 and split BE3, SaKKH-BE3 and split SaKKH-BE3, ABE7.10 and split ABE7.10 (FIG. 7, FIG. 8, FIG. 9).

The results show that BE3 and split BE3, SaKKH-BE3 and split SaKKH-BE3, ABE7.10 and split ABE7.10 have a considerable editing efficiency at the endogenous target.

TABLE 1
oligo design

| | | SEQ ID |
|---|---|---|
| name | sequence | NO.: |
| EMX1 site 1-up | CACCGAAGGACGGCGGCACCGGCGG | 5 |
| EMX1 site 1-dn | AAACCCGCCGGTGCCGCCGTCCTT | 6 |
| EMX1 site2-up | CACCGACTACGTGGTGGGCGCCGAG | 7 |
| EMX1 site2-dn | AAACCTCGGCGCCCACCACGTAGT | 8 |
| RNF2 site1-up | CACCGGTCATCTTAGTCATTACCTG | 9 |
| RNF2 site1-dn | AAACCAGGTAATGACTAAGATGAC | 10 |
| EMX1 site3-up | CACCGCGGATGCACGGTCAGCGCGG | 11 |
| EMX1 site3-dn | AAACCCGCGCTGACCGTGCATCCG | 12 |
| FANCF site1-up | CACCGGCCGTCTCCAAGGTGAAAGC | 13 |
| FANCF site1-dn | AAACGCTTTCACCTTGGAGACGGC | 14 |

TABLE 2
target identification primers

| | | SEQ ID |
|---|---|---|
| name | sequence | NO.: |
| EMX1 site1- | GGAGTGAGTACGGTGTGCTCCCGCGGCT | 15 |
| HITOM-F | GCGACCA | |
| EMX1 site1- | GAGTTGGATGCTGGATGGGGGTGAGGGT | 16 |
| HITOM-R | AGTTGAGCGCC | |
| EMX1 site2- | GGAGTGAGTACGGTGTGCCCCTTCGCAC | 17 |
| HITOM-F | GCAAGCCCAAG | |
| EMX1 site2- | GAGTTGGATGCTGGATGGCGGGGGTGAT | 18 |
| HITOM-R | TACCTGCGTCTCG | |
| RNF2 site1- | GGAGTGAGTACGGTGTGCTATTTCCAGC | 19 |
| HITOM-F | AATGTCTCAGGCT | |
| RNF2 site1- | GAGTTGGATGCTGGATGGGTTTTCATGT | 20 |
| HITOM-R | TCTAAAAATGTATCCCA | |
| EMX1 site3- | GGAGTGAGTACGGTGTGCCTTCGTGAGT | 21 |
| HITOM-F | GGCTTCCCTGCC | |
| EMX1 site3- | GAGTTGGATGCTGGATGGGAAGAAGGAG | 22 |
| HITOM-R | TGCGGGGGCTG | |
| FANCF site1- | GGAGTGAGTACGGTGTGCTCGACCAATA | 23 |
| HITOM-F | GCATTGCAGAGAGGC | |
| FANCF site1- | GAGTTGGATGCTGGATGGGCTGACGTAG | 24 |
| HITOM-R | GTAGTGCTTGAGACC | |

The main advantages of the present invention include:

(1) In the present invention, for the first time, using the existing protein structure information of spCas9 and saCas9 and the split methods, intein-mediated resolution of BE3, SaKKH-BE3, and ABE7.10 were developed, respectively. Split-BE3, SaKKH-BE3 and ABE7.10 have a significant targeted gene mutation efficiency, which is comparable to the targeted gene mutation efficiency of the unsplit BE3, KKH working system, which provides the possibility of packaging into AAV for delivery and promoting its gene widely used in base editing, gene therapy and clinic.

(2) The split single-base gene editing system of the present invention splits BE3, SaKKH-BE3, ABE7.10 into N-terminal and C-terminal, its length is less than packaging limit (4.7 Kb) of the adeno-associated virus (AAV), which can be packaged and delivered with AAV, further expanding the scope of application of the base editor.

The present invention will be further described below in conjunction with specific case. These cases are not only used to illustrate the present invention, but also expande the scope of the present invention. The experimental methods unless specifically stated generally follow conventional conditions such as Sambrook et al. Molecular cloning: the conditions described in the laboratory manual (New York: Cold Spring Harbor Laboratory Press, 1989), or according to the conditions the manufacturer recommended. Unless otherwise stated, percentages and parts are calculated by weight. The experimental materials and reagents involved in the present invention can be obtained from commercially available channels without special instructions.

### Example 1 Comparison of the Mutation Efficiency of Targeting Endogenous Gene Using Unsplit and Split BE3, SaKKH-BE3, ABE7.10

1. Design and Construction of Split-Type Split BE3, SaKKH-BE3, ABE7.10 Plasmid Vector and sgRNA

### 1.1 Design of Split BE3, SaKKH-BE3, ABE7.10 Plasmid

The BE3 and ABE7.10 splits were all fused with SpCas9 (D10A). According to the structure information of SpCas9, splitting occurs between amino acids 573 and 574 of SpCas9; and SaKKH-BE3 is formed by a fusion of SaCas9, According to the structure information of SaCas9, splitting occurs between amino acids 739 and 740 of SpCas9.

1.2 Design Principle of sgRNA:

According to the working principle of CRISPR/Cas9 and BE3, KKH, the human EMX1, RNF2, FANCF target sequences were designed (FIG. 6), the design principle of target sequence sgRNA oligo is as follows:

In CRISPR/Cas9, spCas9 recognizes PAM (NGG) and SaCas9 recognizes PAM (NNNRRT); and at the same time, 20 bases of complementary paired sgRNA are required for targeted binding. The sgRNA uses U6 as the promoter and requires G as the transcription start site. At the same time, U6-SpsgRNA-EF1α-GFP and U6-SasgRNA-EF1α-GFP are ligased into the target site by BbsI enzyme digestion sites. CACCG was needed to be added at 5′ end of sgRNA oligo-up and AAAC was needed to be added at 5′ end of sgRNA oligo-up, the specific sequence is designed as follows.

### 1.3 Construction of the Plasmid in 1.2

Synthesis of sgRNA Oligo.

1.3.1 Dissolving oligo in pure water to a final concentration of 100 μM.
1.3.2 Annealing. 10 μL of each of the two complementary oligos were mixed, and put in a boiling water bath for 5 min, then cooled naturally to room temperature, about 2 hours.
1.3.3 ligased. U6-SpsgRNA-EF1α-GFP (BbsI) and U6-SasgRNA-EF1α-GFP (BbsI) were digested with BbsI and the vectors and annealed oligo were ligated and reacted according to the following reaction system.

Take Biyuntian's Rapid DNA ligation kit (D7006) as an example.

| | |
|---|---|
| annealed oligo | 1 μL |
| precut vector | 1 μL (10-50 ng) |
| T4 DNA Ligase | 1 μL |
| 10x T4 DNA Ligase buffer | 1 μL |
| H2O | add to 10 μL |

After ligased at room temperature for 60 min, 5 μL mixture was transformed into 50 μL of competent bacteria, coated with kanamycin-resistant plates, and incubated overnight at 37° C.

1.3.4 Two clones was picked from the overnight culture plate, inoculated in a 4-5 mL medium, shaked at 37° C., incubated overnight at 220 r/min.
1.3.5 After shaking culture overnight, the plasmids were extracted and verified by hU6 sequencing, sequencing the correct plasmid.

### 2. Comparison of Targeted Endogenous Gene Mutation Efficiency of Unsplit and Split BE3, SaKKH-BE3 and ABE7.10.

### 2.1 Plasmid Transfection

Day 1 Seeding a 24-Well Plate with 293T Cells
2.1.1 The HEK293T cells were digested and seeded a 24-well plate at 2.0×10⁵ cell/well.

Note: After the cells were recovered, they generally need to be passaged twice before being used for transfection experiments.

### Day 2 Transfection

2.1.2 The state of cells in each well was observed.

Note: The cell density before transfection should be 80%-95%, and the condition is normal. 2.1.3 To ensure the accuracy of the data and the repeatability of the experiment, the plasmid was diluted with sterile water. Diluting the plasmid concentration of each group to be consistent, or ensure that the volume of plasmid samples between the groups was the same.

The group settings were as follows:

Blank: blank control, including only cultured cells and culture medium;

The treatment groups were:

That is the combination of the following was used to transfect 293T,

BE3: U6-sgRNA-EF1α-GFP=750 ng:250 ng,

BE3-N: BE3-C: U6-sgRNA-EF1α-GFP=375 ng:375 ng:250 ng

SaKKH-BE3: U6-sgRNA-EF1α-GFP=750 ng:250 ng,

SaKKH-BE3-N:SaKKH-BE3-C:U6-sgRNA-EF1α-GFP=375 ng:375 ng:250 ng

ABE7.10: U6-sgRNA-EF1α-GFP=750 ng:250 ng,

ABE7.10-N: ABE7.10-C: U6-sgRNA-EF1α-GFP=375 ng:375 ng:250 ng

2.1.4 Adding DMEM (no serum, no antibiotics) to the 1.5 mL EP tube.
2.1.5 Adding the DNA plasmid to the EP tube in step (4) and mixing it well.
2.1.6 Adding PEI to the EP tube in the previous step, mixing it well, and letting stand at room temperature for 20 minutes.
2.1.7 Adding the transfection mix to the 24-well plate. Gently tapping the 24-well plate to mix well.
2.1.8 After culturing at 37° C., 5% CO₂ for 72 h, GFP-positive cells were sorted by flow cytometry.

### Day 5

2.1.9 After 72 h, GFP positive cells were sorted by flow cytometry.
2.1.10 Extracting the genomic DNA of the sorted GFP-positive cells with Tiangen Cell Genome Extraction Kit (DP304), and performing PCR using the first round of PCR identification primers (Table-2) according to the instructions of the HITOM kit (Nuohe Zhiyuan Item No.: PT026). After the second round of PCR library construction is completed, high-throughput sequencing were performed. Comparing the editing efficiency of endogenous targets for BE3 and split BE3, SaKKH-BE3 and split SaKKH-BE3, ABE7.10 and split ABE7.10 (FIG. 7, FIG. 8, FIG. 9).

The results show that BE3 and split BE3, SaKKH-BE3 and split SaKKH-BE3, ABE7.10 and split ABE7.10 have a considerable editing efficiency at each endogenous target.

### Comparative Example 1

The method of Example 1 was used, except that co-transformation of BE3-N terminal or C terminal with the sgRNA-expressing plasmid, and co-transformation of SaKKH-BE3-N terminal or C terminal with the sgRNA-expressing plasmid and co-transformation of ABE7.10-N terminal or C terminal with the sgRNA-expressing plasmid.

The result shows that none of the endogenous gene mutations are detected.

### Comparative Example 2

The method of Example 1 was used, except that there was no corresponding intein at BE3-N, C-terminal, no corresponding intein at SaKKH-BE3-N, C-terminal, ABE7.10-N or C-terminal, and they were co-transformed with sgRNA plasmid.

The result shows that none of the endogenous gene mutations are detected.

### Comparative Example 3

The method of Example 1 was used, except that BE3-C and SaKKH-BE3-C were not fused with glycosidase inhibitors and co-transformed with sgRNA plasmids.

The result shows that the endogenous gene mutation efficiency is lower, about 10%, and there are also lower unexpected mutations, namely mutation of cytosine to adenine, guanine.

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

1. A nucleic acid construct combination comprising a first nucleic acid construct and a second nucleic acid construct, wherein the first nucleic acid construct has a structure of Formula I from 5′-3′:
P1-X1-X2-X3-Z-X4 (I);
wherein P1 is a first promoter sequence;
X1 is a coding sequence of cytosine deaminase or a coding sequence of adenosine deaminase;
X2 is an optional linker sequence;
X3 is a coding sequence of the N-terminal fragment of Cas9 nuclease or a coding sequence of the N-terminal fragment of SaKKH nuclease (Cas9n-N or SSaKKH-N);
Z is a coding sequence of the N-terminal fragment of a first fusion peptide;
X4 is a polyA sequence;
the second nucleic acid construct has a structure of Formula II from 5′-3′:
P2-Z-Y1-Y2-Y3 (II);
wherein P2 is a second promoter sequence;
Z is a coding sequence of the C-terminal fragment of a first fusion peptide;
Y1 is a coding sequence of the C-terminal fragment of Cas9 nuclease or a coding sequence of the C-terminal fragment of SSaKKH nuclease (Cas9n-C or SSaKKH-C);
Y2 is a coding sequence of UGI or none;
Y3 is a polyA sequence;
and each “-” is independently a bond or a nucleotide linker sequence.

2. The nucleic acid construct combination of claim 1, wherein the first promoter and the second promoter are each independently selected from the group consisting of: a CAG promoter, CMV promoter, and a combination thereof.

3. The nucleic acid construct combination of claim 1, wherein the Cas9 nuclease is selected from the group consisting of Cas9, Cas9n, and a combination thereof.

4. The nucleic acid construct combination of claim 1, wherein the origin of the X3 element is selected from the group consisting of Streptococcus pyogenes, Staphylococcus aureus, and a combination thereof.

5. The nucleic acid construct combination of claim 1, wherein the length of the first nucleic acid construct is ≤4.7 kb, preferably, ≤4.5 kb, and more preferably, 3.0-4.5 kb.

6. The nucleic acid construct combination of claim 1, wherein the length of the second nucleic acid construct is ≤4.7 kb, preferably, ≤4.5 kb, and more preferably, 3.0-4.5 kb.

7. A vector combination, comprising a first vector and a second vector, the first vector contains a first nucleic acid construct, the second vector contains the second nucleic acid construct, the first nucleic acid construct and the second nucleic acid construct are as defined in claim 1.

8. A genetically engineered cell wherein the cell is transformed by the construct of claim 1.

9. A method for gene editing in a cell, comprising the steps of:
(i) providing a cell and a first vector and a second vector, wherein the first vector contains a first nucleic acid construct, the second vector contains a second nucleic acid construct, the first nucleic acid construct and the second nucleic acid construct are as defined in claim 1, and both the first vector and the second vector are viral vectors;
(ii) infecting the cell with the viral vector, thereby performing gene editing in the cell.

10. A kit, comprising:
(a1) a first container, and a first vector located in the first container; and
(b1) a second container, and a second vector located in the second container.

11. A genetically engineered cell wherein the cell is transformed or transfected by the vector combination of claim 7.


SEQUENCE LISTING (24 sequences)

SEQ ID NO: 1 - protein, 958 aa, Streptococcus pyogenes
MDYKDHDGDYKDHDIDYKDDDDKMAPKKKRKVGIHGVPAASSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 2 - protein, 1125 aa, Staphylococcus aureus
MDYKDHDGDYKDHDIDYKDDDDKMAPKKKRKVGIHGVPAASSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESGKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAECLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 3 - protein, 480 aa, Staphylococcus aureus
MIKIATRKYLGKQNVYDIGVERDHNFALKNGFIASSMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKGGSPKKKRKVSSDYKDHDGDYKDHDIDYKDDDDKSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 4 - protein, 939 aa, Streptococcus pyogenes
MIKIATRKYLGKQNVYDIGVERDHNFALKNGFIASCFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKVGSYPYDVPDYA

SEQ ID NO: 5 - DNA, 25 bp, artificial sequence
  synthesized
  misc_feature: EMX1 site1-up
caccgaaggacggcggcaccggcgg

SEQ ID NO: 6 - DNA, 24 bp, artificial sequence
  synthesized
  misc_feature: EMX1 site1-dn
aaacccgccggtgccgccgtcctt

SEQ ID NO: 7 - DNA, 25 bp, artificial sequence
  synthesized
  misc_feature: EMX1 site2-up
caccgactacgtggtgggcgccgag

SEQ ID NO: 8 - DNA, 24 bp, artificial sequence
  synthesized
  misc_feature: EMX1 site2-dn
aaacctcggcgcccaccacgtagt

SEQ ID NO: 9 - DNA, 25 bp, artificial sequence
  synthesized
  misc_feature: RNF2 site1-up
caccggtcatcttagtcattacctg

SEQ ID NO: 10 - DNA, 24 bp, artificial sequence
  synthesized
  misc_feature: RNF2 site1-dn
aaaccaggtaatgactaagatgac

SEQ ID NO: 11 - DNA, 25 bp, artificial sequence
  synthesized
  misc_feature: EMX1 site3-up
caccgcggatgcacggtcagcgcgg

SEQ ID NO: 12 - DNA, 24 bp, artificial sequence
  synthesized
  misc_feature: EMX1 site3-dn
aaacccgcgctgaccgtgcatccg

SEQ ID NO: 13 - DNA, 25 bp, artificial sequence
  synthesized
  misc_feature: FANCF site1-up
caccggccgtctccaaggtgaaagc

SEQ ID NO: 14 - DNA, 24 bp, artificial sequence
  synthesized
  misc_feature: FANCF site1-dn
aaacgctttcaccttggagacggc

SEQ ID NO: 15 - DNA, 35 bp, artificial sequence
  synthesized
  misc_feature: primer
ggagtgagtacggtgtgctcccgcggctgcgacca

SEQ ID NO: 16 - DNA, 39 bp, artificial sequence
  synthesized
  misc_feature: primer
gagttggatgctggatgggggtgagggtagttgagcgcc

SEQ ID NO: 17 - DNA, 39 bp, artificial sequence
  synthesized
  misc_feature: primer
ggagtgagtacggtgtgccccttcgcacgcaagcccaag

SEQ ID NO: 18 - DNA, 41 bp, artificial sequence
  synthesized
  misc_feature: primer
gagttggatgctggatggcgggggtgattacctgcgtctcg

SEQ ID NO: 19 - DNA, 41 bp, artificial sequence
  synthesized
  misc_feature: primer
ggagtgagtacggtgtgctatttccagcaatgtctcaggct

SEQ ID NO: 20 - DNA, 45 bp, artificial sequence
  synthesized
  misc_feature: primer
gagttggatgctggatgggttttcatgttctaaaaatgtatccca

SEQ ID NO: 21 - DNA, 40 bp, artificial sequence
  synthesized
  misc_feature: primer
ggagtgagtacggtgtgccttcgtgagtggcttccctgcc

SEQ ID NO: 22 - DNA, 39 bp, artificial sequence
  synthesized
  misc_feature: primer
gagttggatgctggatgggaagaaggagtgcgggggctg

SEQ ID NO: 23 - DNA, 43 bp, artificial sequence
  synthesized
  misc_feature: primer
ggagtgagtacggtgtgctcgaccaatagcattgcagagaggc

SEQ ID NO: 24 - DNA, 43 bp, artificial sequence
  synthesized
  misc_feature: primer
gagttggatgctggatgggctgacgtaggtagtgcttgagacc